(19) United States
(12) Patent Application Publication
(10) Pub. No.: US 2024/0293646 A1
(43) Pub. Date: Sep. 5, 2024

(54) SAFETY CATHETER ASSEMBLY

(71) Applicant: SMITHS MEDICAL ASD, INC., PLYMOUTH, MN (US)

(72) Inventors: Christopher D Roehl, New Hartford, CT (US); Thomas T Koehler, Pelzer, SC (US)

(73) Assignee: SMITHS MEDICAL ASD, INC., PLYMOUTH, MN (US)

(21) Appl. No.: 18/571,339

(22) PCT Filed: Jul. 25, 2022
(86) PCT No.: PCT/US2022/038118

§ 371 Date: Dec. 18, 2023

(30) Foreign Application Priority Data
Jul. 30, 2021 (US) 63227508

(51) Int. Cl. A61M 25/06

(52) U.S. Cl. CPC A61M 25/0618; A61M 25/0606

(57) ABSTRACT

A safety catheter assembly has a clip guard that covers and protects the needle clip from the time that the catheter assembly is shipped out in the ready state, through its use where the distal tip of the needle is entrapped in the needle clip after use, and to when the clip guard and the safety needle clip housed therein are removed from the catheter hub. A passive safety system of the needle clip couples the catheter hub and the needle housing in the ready state and during the use of the catheter assembly, and releases the catheter hub from the needle housing once the contaminated needle tip is captured in the needle clip. A wiper provided to the catheter assembly wipes blood from the needle to prevent exposure of the contaminated blood. The wiper may also act to retain a septum in the catheter hub. The clip guard may be adapted to be used with a catheter hub having a multiuse seal member.

### FIELD OF THE INVENTION

The present invention relates to safety catheter devices that may include a closed system catheter (CSC) assembly having a needle tip protection guard housing a passive release mechanism that shields the sharp distal tip of the needle of the device from when the device is ready to be used to the disposal of the device after use, and a mechanism adapted to remove blood that may have adhered to the needle during use.

### BACKGROUND OF THE INVENTION

A conventional catheter assembly typically has a catheter hub and a catheter tube extending distally thereof, and a needle assembly mounted together in an over-the-needle fashion. The needle assembly typically includes a needle hub or support and a needle cannula extending distally from the needle hub. A closed system catheter (CSC) is a catheter assembly that has a sideport at the catheter hub where a tubing from the sideport may be coupled to a fluid store device such as a syringe or a fluid pump. The sideport for a CSC assembly is an external port conventionally angled backward toward the proximal end of the catheter, with the tubing extending from the non-catheter hub end of the external port. In a ready to use position, a needle cannula attached to a needle hub of the needle assembly extends through the catheter tube to expose its sharp distal tip distally beyond the distal end of the catheter tube. The sharp distal tip of the needle cannula is used to penetrate the tissue of the patient for insertion of the catheter tube within the vasculature system of a patient. Once the catheter tube is disposed correctly within the vasculature, the needle cannula is withdrawn from the catheter assembly so that a fluid path is established between the fluid store and the vein or artery of the patient, via the sideport and the interior cavity of the catheter hub. A needle tip protector may be provided in the catheter assembly to capture at least the sharp distal tip of the needle cannula, if not the entire cannula, after use.

The catheter hub typically has an open proximal end adapted to receive a male luer taper into the interior cavity of the catheter hub to establish a fluid connection between the patient's vasculature and the luer taper. The proximal end may also be provided with external ears, flange or the like to secure the luer taper in the catheter hub, for example when the luer taper is coupled with a male luer lock collar or nut to form part of a male luer lock connector of an administration set or the end of a syringe, or the like. Under normal conditions, after withdrawal of the needle cannula and before a luer taper is inserted into the catheter hub, blood immediately starts flowing through the catheter tube and into the interior cavity of the catheter hub. For a CSC assembly, a septum may br provided in the catheter hub to prevent the blood from back flowing to the proximal luer end of the catheter hub.

Fluid such as for example contaminated blood that may have adhered to the distal portion of the needle conceivably may be exposed to the environment. In use, the distal tip of the needle cannula, which extends beyond the distal end of the catheter, is inserted into the vein or artery of the patient. Once the catheter is correctly placed, the needle cannula is removed. The distal tip of the needle, insofar as it was inserted into for example the vein of the patient, is exposed to the blood of the patient. Blood therefore will most likely adhere to the distal portion of the needle. There is always the chance that the contaminated blood adhered to the needle would be exposed to the environment.

### SUMMARY OF THE PRESENT INVENTION

The present invention safety catheter assembly provides an improvement over the prior art safety catheter assemblies by having a needle tip protector, or needle clip, housed in a needle tip protector guard, or clip guard, that covers the proximal portion of the catheter hub from the ready state to when the needle has been withdrawn from the patient and entrapped in the needle clip. A wiper mechanism, or simply wiper, is preferably provided at the proximal end of the catheter hub to wipe off blood that may have adhered to the needle due to the distal portion of the needle having inserted into the patient. The wiper mechanism acts to remove blood from the needle as the needle is withdrawn from the catheter hub into the needle tip protector. A wiper mechanism fitted to the opened proximal end of the catheter hub also acts to close off the proximal end of the catheter hub in addition to wiping off the blood that has adhered to the needle. All the while, the needle clip remains covered by the clip guard so that neither the tip of the needle nor the needle clip is exposed to the environment. The wiper mechanism may also be used to retain a seal member or septum provided in the internal cavity of the catheter hub. The needle clip is a passive mechanism that couples the catheter hub and the clip guard together and traps the distal tip after use. The needle clip has fingers or catches that grasp or grip at least one portion of at least one outwardly extending flange at the proximal end of the catheter hub before the distal tip is fully entrapped within the needle clip. Once the distal tip of the needle is trapped in the needle clip, the fingers of the needle clip would release its grip of the flange of the catheter hub to disengage the clip guard from the catheter hub.

The present invention is directed to a catheter assembly comprising: a catheter hub having a body defined by a wall having a distal end and an opened proximal end having a flange, and an internal cavity defined by an inner surface of the wall between the distal end and the opened proximal end; a catheter having a distal end extending from the distal end of the catheter hub along a longitudinal axis; a wiper member including a distal section having a cross dimension configured to fit into the opened proximal end of the catheter hub distal of a proximal end of the seal member and a proximal section having a cross dimension larger than the cross dimension of the distal section; a needle housing having a guard having an open distal end and a needle hub; a needle having a proximal end attached to the needle hub and a sharp distal tip extending away from the open distal end of the housing along the longitudinal axis, the needle slidably extending along the catheter so that its distal tip extends past the distal end of the catheter when the catheter assembly is in a ready to use position; a needle tip protector slidably positioned along the needle; wherein the opened distal end of the guard opens to a chamber configured to house the needle tip protector.

The present invention is further directed to a wiper mechanism provided between the catheter hub and the clip guard to wipe off excess blood adhered to the needle as the needle is being removed form the catheter hub to prevent accidental exposure of contaminated blood to the environment and potential harm to the user or clinician. The wiper mechanism may be provided to the proximal end of the catheter hub and is configured to operate with the needle clip.

The invention furthermore is directed to the needle clip being non-removably housed in the clip guard, which is configured to cover the proximal portion of the catheter hub to ensure that the distal portion of the needle is enclosed throughout the life and use of the catheter assembly.

### BRIEF DESCRIPTION OF THE FIGURES

The present invention will become apparent and the invention itself will be best understood with reference to the following description of the invention taken in conjunction with the accompanying drawings, wherein:

FIG. 1 is a perspective view of an exemplar embodiment of a sideport safety catheter assembly of the instant invention;

FIG. 2 shows the sheath having been removed from the sideport catheter assembly;

FIG. 3 is an enlarged cross-sectional view of the catheter hub and a distal portion of the needle housing of the sideport catheter assembly;

FIG. 4a is a side view of the exemplar sideport catheter assembly;

FIG. 4b is a cross-sectional view of the FIG. 4a sideport catheter assembly;

FIG. 4c is a cross-sectional plan view of the FIG. 4a sideport catheter assembly;

FIG. 5 shows an exemplar embodiment of the present invention where the needle clip and an exemplar catheter hub are coupled to each other;

FIG. 6 shows the exemplar needle clip no longer coupled to the exemplar catheter hub;

FIG. 7a is a perspective view of an exemplar catheter assembly;

FIG. 7b is a perspective of the exemplar catheter assembly and semi-transparent view of the clip guard shown in FIG. 7a;

FIG. 8a is a side view illustration of the needle clip and an exemplar catheter hub coupled to each other;

FIG. 8b is another view showing the needle clip and the catheter hub coupled to each other;

FIG. 9 shows a needle clip housed in a semi-transparent clip guard disengaged from the catheter hub;

FIG. 10a is another view showing the needle clip in engagement with the catheter hub while covered by the clip guard;

FIG. 10b is another view of the catheter hub coupled to the needle clip housed in the clip guard, and the clip guard circumferentially covering the proximal portion of the catheter hub;

FIG. 11a is a perspective view showing the interaction between the needle clip and the catheter hub;

FIG. 11b is a side view of the catheter coupled to the needle clip;

FIG. 12a is another perspective view of the catheter hub and the needle clip coupled to each other;

FIG. 12b shows yet another perspective view of the catheter hub and the needle clip coupled to each other;

FIG. 13a shows the needle clip of FIG. 5-6 without the catheter hub;

FIG. 13b is another view of the exemplar needle clip of FIG. 13a;

FIG. 14 is a perspective view of an exemplar catheter assembly embodiment where the catheter hub has a wiper member and is received into the distal portion of the clip housing;

FIG. 15 is an enlarged view of the proximal portion of the catheter hub and a semi-transparent view of the clip guard showing the inter-relationship among the needle clip, the wiper member and the catheter hub for the safety catheter assembly embodiment shown in FIG. 14;

FIG. 16 is another view of FIG. 15 from a different perspective;

FIG. 17 is a cross-sectional view of the proximal portion of the catheter hub, the wiper member and the needle clip covered by the distal portion of the semi-transparent clip guard;

FIG. 18 is another perspective view of inventive components shown in FIG. 17;

FIG. 19 is a semi-transparent perspective view of another embodiment of the inventive catheter assembly;

FIG. 20 is a cross-sectional plan view of the FIG. 19 catheter assembly;

FIG. 21 is a cross-sectional side view of the FIG. 19 catheter assembly; and

FIG. 22 is an enlarged view showing the interaction among the catheter hub, the needle clip and the clip guard of the FIG. 19 catheter assembly.

### DETAILED DESCRIPTION OF THE INVENTION

For the discussion below, it should be appreciated that the term “distal”, as used herein, refers to the direction along an axis that lies parallel to a needle cannula of a safety catheter assembly, that is closest to the subject during catheter insertion. Conversely, the term “proximal,” as used herein, refers to the direction lying along the axis parallel to the needle cannula that is further away from the subject when the catheter is inserted into the vein of the subject, opposite to the distal direction. Other terms that may also be used to refer to the distal end and proximal end of the safety catheter device may include “patient end” and “user end”, respectively. Also, for the sake of simplicity, the catheter assembly device, or peripheral intravascular catheter (PIVC) assembly, and the closed system catheter (CSC) assembly may be referred to as “catheter assembly” or “safety catheter”, the needle tip protector as “safety needle clip” or simply “safety clip” or “needle clip”, the wiper member or mechanism as “nose wiper” or simply “wiper”, and the needle guard assembly that overlies the proximal portion of the catheter hub and houses the needle clip to isolate the needle clip from the environment simply as “clip guard”.

The following US applications and patents, assigned to the same assignee as the present application, relate to safety catheters/catheter assemblies: application Ser. Nos. 15/733,439, 15/435,700, U.S. Pat. Nos. 8,652,104, 10,080,867, 10,548,522 and 10,675,440. The respective disclosures of the '439 and '700 applications and the '104, '867, '522 and '440 patents are incorporated by reference to the disclosure of the present application.

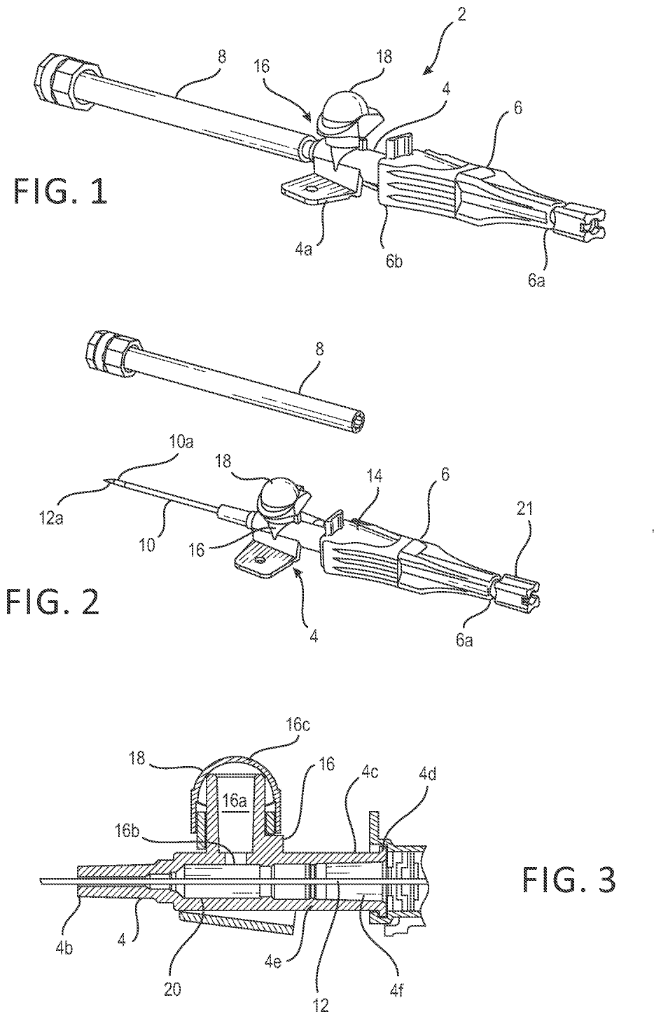

With reference to FIGS. 1, 2 and 4a-4c, an exemplar sideport safety catheter assembly, or catheter assembly 2 as discussed herein, is shown to have a catheter hub 4, a needle housing 6 and a sheath 8 that covers a catheter 10 that extends distally from catheter hub 4. A needle 12 has a sharp distal tip 12a that extends beyond the distal end 10a of catheter 10. A non-patient end 12b of needle 12 is fixedly attached to a needle hub 6c of needle housing 6. The needle shaft defines a lumen and may have a side orifice proximate to its distal tip as is conventionally know to enable fluid to flow into the lumen and egress at the orifice into a space defined between the outer wall of the needle and the inner wall of the catheter and may flow into a flash chamber when the distal end of the needle is inserted into a patient. Needle housing 6 has an opened distal end adapted to receive a needle tip protector guard, or clip guard 14 hereinafter. As shown in FIGS. 1, 2 and 4c, catheter hub 4 has a pair of wings 4a for securing the catheter hub to the skin of the patient as is conventionally known. A sideport hub, or simply sideport 16, integrally extends transversely from catheter hub 4. Sideport 16 is shown being covered by a snap on cap 18. A flash plug 21 is attached to the proximal end 6a of needle housing 6 to enable a user or clinician to monitor blood at the flash chamber of needle housing 6 to determine whether the needle, and the catheter that fits over the needle, are correctly placed into the vasculature of the patient as conventionally known.

FIG. 3 is an enlarged cross-sectional view of the catheter hub. As shown, catheter hub 4 has a distal end 4b and a proximal end 4c. One continuous flange 4d or multiple protuberances or flanges 4d integrally extend transversely from proximal end 4c. Catheter hub 4 is shown to have a body defined by a wall 4e that forms an internal cavity 4f. Integrally extending outwardly from the main body of catheter hub 4 is a sideport hub 16, which has a passage or bore 16a that extends into internal cavity 4f via an opening 16b at wall 4e to establish a fluid communication path between the internal cavity of the catheter hub 4 and sideport 16. A cylindrical seal member 20 which may be made from a flexible resealable elastomeric material such as silicone or polyisoprene is fitted in the portion of internal cavity 4f to block opening 16b. As is conventionally known, with cap 18 removed, the open end 16c of sideport hub 16 may be connected to a fluid store such as a syringe or a fluid injection pump, possibly by means of a tubing, so that fluid under pressure may be injected into sideport 16. Seal member 20 is responsive to a given pressure so that when the fluid injected into sideport 16 exceeds the given pressure, seal member 20 opens to establish a through fluid path between fluid store and internal cavity 4f of catheter hub 4, and from there through catheter 10 to the vein of the patient.

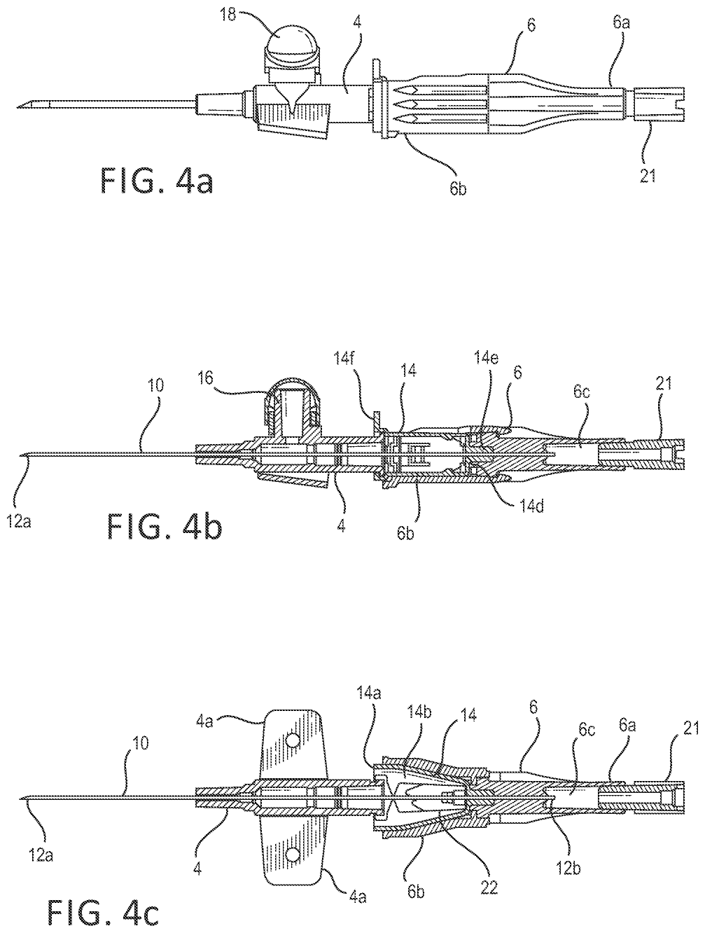

With reference to FIGS. 4a-4c, the sideport catheter assembly is shown to have a proximal portion of its catheter hub 4 covered by its needle tip protector guard, or simply clip guard 14. A substantial portion of clip guard 14 is shown to be received in distal portion 6b of needle housing 6. The sideport catheter assembly shown in FIGS. 4a-4c is in a ready to use position, a ready to use state or simply a ready state.

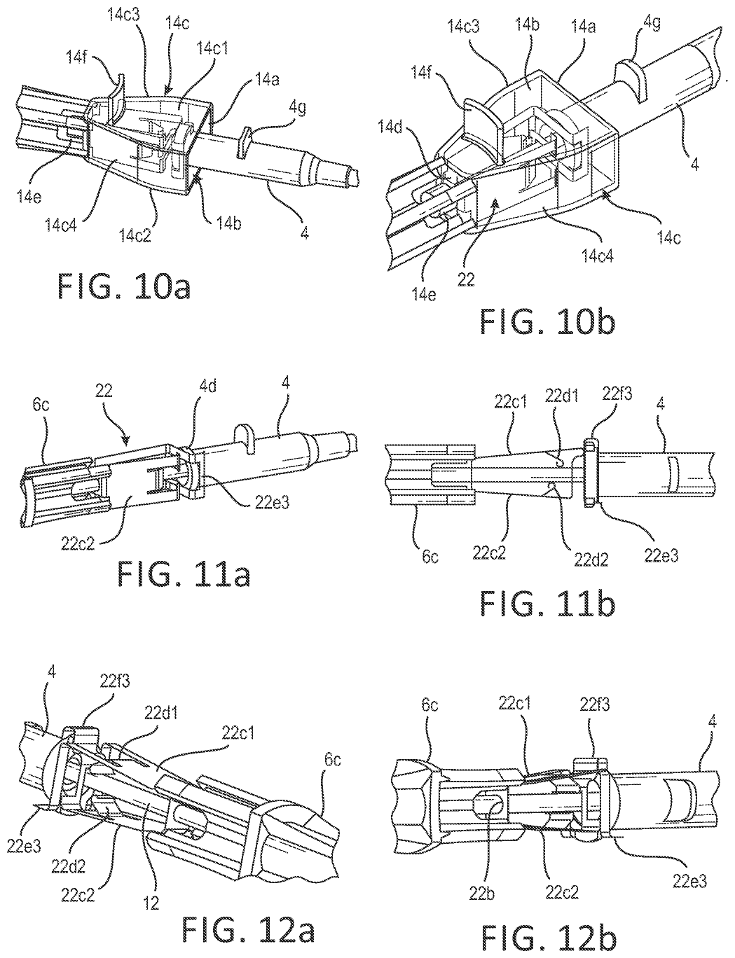

As shown in FIGS. 9 and 10a-10b, clip guard 14 has an opened distal end 14a that opens into a chamber 14b defined by a body 14c having a top wall or surface 14c1, a bottom wall 14c2, and two side walls 14c3 and 14c4 that taper from the a flat surface proximal from distal end 14a to a base 14d that includes a hollow shaft 14e. An upright 14f rises from top surface 14c1 to assist the user or clinician to direct the movement of the needle into the patient. It should be appreciated that the embodiment of the catheter assembly shown in FIGS. 10a-10b is not the sideport catheter assembly shown in FIGS. 1-3.

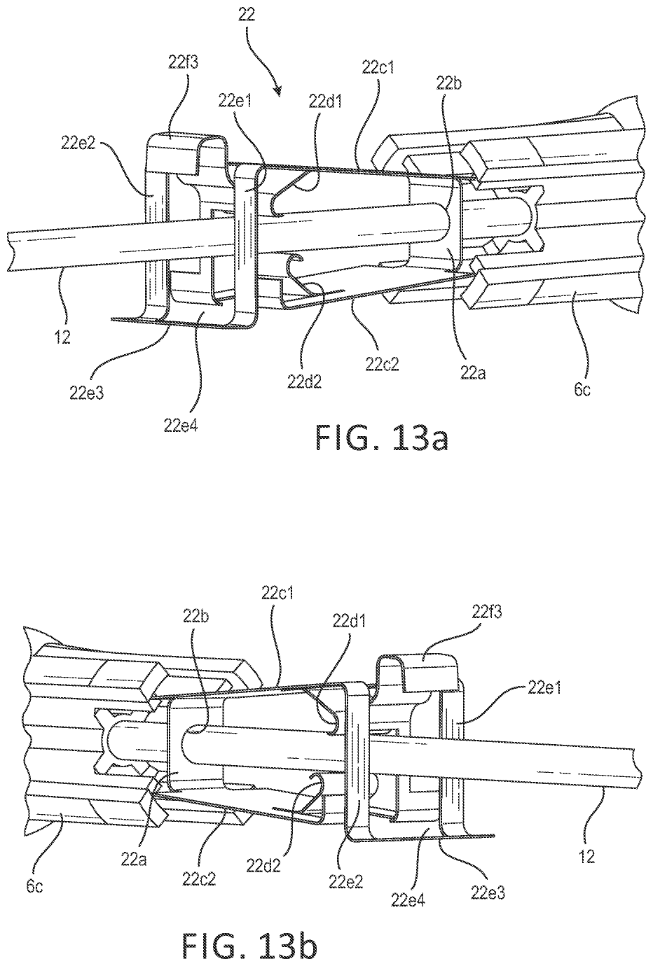

A needle tip protector, or needle clip 22, is non-removably housed in chamber 14b of clip guard 14. An exemplar embodiment of needle clip 22 is shown in FIGS. 13a-13b. As shown in the ready state of the catheter assembly, needle clip 22 is slidable along needle 12, which lies along the longitudinal axis of the catheter assembly. Needle clip 22 may be made from a metallic material such as stainless steel but with a thickness sufficient to enable the needle clip to be relatively rigid but yet flexible for those parts that require flexibility or bendability when biased. In particular, needle clip has a base 22a that has an opening 22b. Opening 22b is configured to have a dimension that enables needle clip 22 to slide freely therealong but prevent a needle feature 12c (FIG. 6) such as a protuberance or an enlarged portion of the needle proximate to distal tip 12a to pass through. This is so that when needle feature 12c makes contact with opening 22b, further proximal movement of needle 12 relative to catheter hub 4 would cause needle clip 22 to move in unison with needle 12 in the proximal direction.

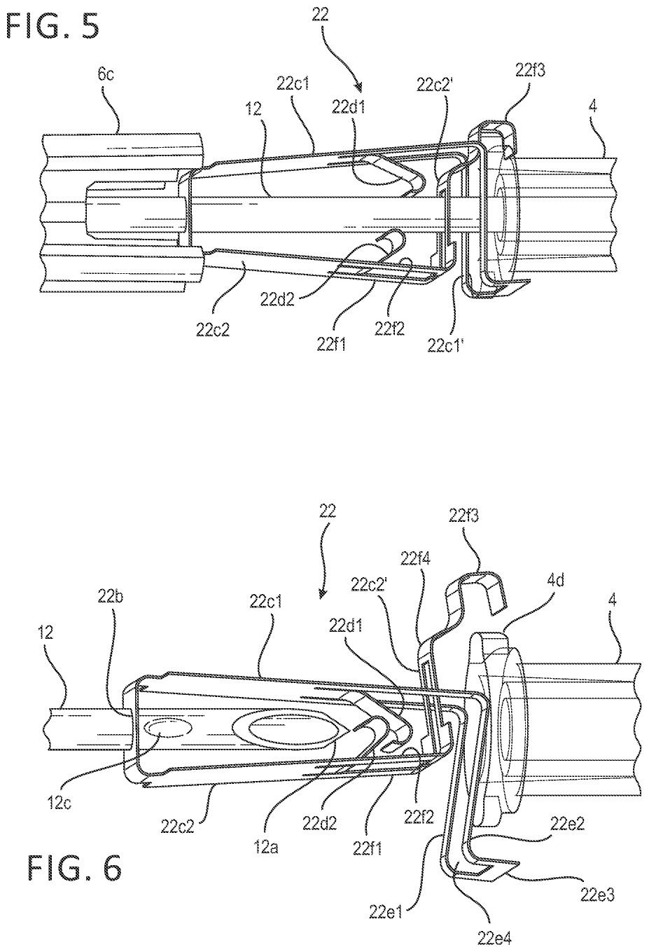

With reference to FIGS. 5-6 and 13a-13b which do not show the clip guard, needle clip 22 is shown to have two arms 22c1 and 22c2 that extend substantially transversely in the distal direction from opposite ends of base 22a. Respective mid-sections 22d1 and 22d2 of the arms 22c1 and 22c2 are bent inwardly toward the center of needle clip 22, with the tip ends of those mid-sections curved to form corresponding catches or barriers. The mid-sections henceforth are referred to as barriers 22d1 and 22d2. As per shown in FIG. 6, needle feature 12c is in stop contact with opening 22b at the base of needle clip 22. Thus, distal tip 12a of needle 12 is entrapped in needle clip 22, with barriers 22d1 and 22d2 returning to their natural positions since they are no longer biased by needle 12. As shown, barrier 22d1 is distal to barrier 22d2, with distal tip 12a of needle 12 proximal to both barriers 22d1 and 22d2. FIG. 6 shows the catheter assembly in the safe state as the contaminated distal tip of the needle is trapped inside needle clip 22.

Further with reference to FIGS. 5-6 and 13a-13b, the respective portions of arms 22c1 and 22c2 are configured to have slots defined by parallel rails. In particular, parallel rails 22e1 and 22e2 extend from the mid-section of arm 22c1 to a cross end 22e3 that connects the rails to define a slot 22e4 with a width that allows needle 12 to pass through. As shown, rails 22e1 and 22e2 extend distally coplanarly with arm 22c1 to a distance that meets the proximal end of catheter hub 4. Rails 22e1 and 22e2 are then bent slightly off at right angle downwardly to form a section adapted to contact the proximal end surface of catheter hub 4. The end portion of rails 22e1 and 22e2 are further bent in the distal direction substantially transverse to the section adapted to contact the proximal end of catheter hub 4 such that end 22e3 is configured to be adapted to press against the flange 4d of catheter hub 4 when barriers 22d1 and 22d2 are biased by needle 12 in the ready state, as per shown in FIG. 5. Similarly, arm 22c2 is bent in a reverse manner such that rails 22f1 and 22f2 are bent upwardly at a slightly off right angle from the longitudinal portion of arm 22c2 to form a mid-section that defines a slot 22f4 with space sufficient to allow needle 12 to pass through at a distance proximal from rails 22e1 and 22e2. The distal section 22c2′ of arm 22c2 is configured in the shape of a hook 2f3 adapted to fittingly grasp the part of flange 4d opposite to the part of the flange held by end 22e3 of arm 22c1 when needle clip 22 is coupled to catheter hub 4 in the ready state, as per shown in FIG. 5. Hook 22f3 and distal section 22c2′ of arm 22c2 pass though slot 22e4 of arm 22c1.

Thus, as shown in FIGS. 5 and 13a-13b, when the catheter assembly is in the ready state, barriers 22d1 and 22d2 are biased by needle 12 away from the longitudinal axis of the catheter assembly. Needle 12 also passes slots 22f4 and 22e4 of arms 22c2 and 22c2, respectively, into catheter hub 4. With barriers 22d1 and 22d2 biased outwardly, transverse portions 22c1′ and 22c2′ would move in opposite directions inwardly toward the longitudinal axis such that hook 22f3 catches flange 4d and end 22e3 presses against flange 4d of catheter hub 4 such that needle clip 22 and catheter hub 4 are coupled to each other. It should be appreciated that instead of one flange, there may be multiple flanges or protuberances provided at the proximal end of catheter hub 4. In which case, hook 22f3 and end 22e3 would grasp opposite flanges. It should further be appreciated that hook 22f3 and end 22e3 are meant to illustrate grasping mechanisms that may be used. In practice, the arms each may have a hook at their distal end, or some other finger mechanisms that allow the needle clip to grippingly hold catheter hub 4. Hook 22f3 and end 22e3 as discussed above may be referred to henceforth as grippers. It should furthermore be appreciated that needle clip 22 as described above may be adapted to be used for safety catheter assembles that do not have a sideport hub as per shown in FIGS. 1-3, and CSC assemblies that have an external port that angles towards the open proximal end of the catheter hub and a tubing fixedly extending from the non-catheter hub end of the external port.

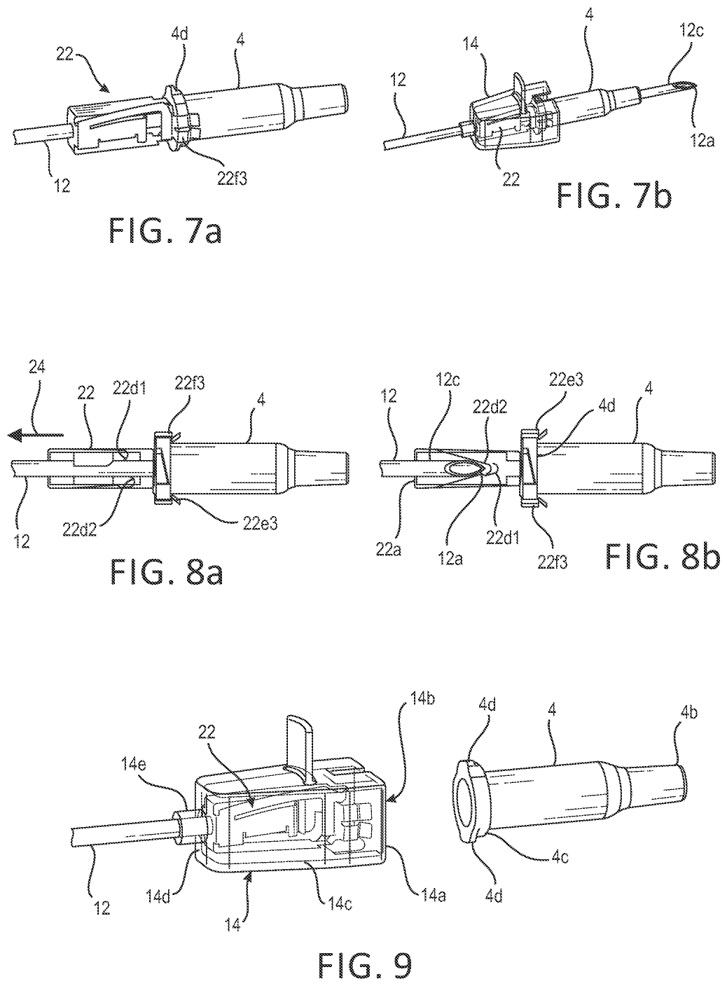

FIG. 7a shows needle clip 22 and catheter hub 4 coupled to each other by means of dual finger hooks 22f3 instead of the single hook discussed above and shown in FIGS. 5-6. Also, the end 22e3 of the FIG. 5-6 embodiment has been replaced by dual finger hooks 22e3. Functionally, the finger hooks in FIGS. 7a-7b, 8a-8b and 9 operate in the same manner as the grippers discussed with reference to FIGS. 5-6. FIG. 7b shows needle clip 22 housed inside clip guard 14, which distal portion superposes over so as to cover and protect the proximal portion of catheter hub 4. Of course, being housed inside clip guard 14, needle clip 22 is also covered and protected by clip guard 14. For illustration purpose, the catheter is removed from the distal end of catheter hub 4 so that a better view may be had for needle feature 12c and distal tip 12a of needle 12. FIGS. 7a-7b show the catheter assembly in a ready state, or a ready to use position.

FIGS. 8a-8b illustrate the activated state of the needle assembly. In FIG. 8a, needle 12 is shown to have been withdrawn in the proximal direction as indicated by directional arrow 24. it is assumed that the distal tip of needle 12 has moved into the internal cavity of catheter hub 4. At this point, since barriers 22d1 and 22d2 of needle clip 22 continue to be biased by needle 12, hooks 22f3 and 22c3 remain in the position as shown so that catheter hub 4 and needle clip 22 remain coupled to each other.

FIG. 8b shows needle 12 having been further withdrawn in the proximal direction such that distal tip 12a is positioned proximal of barriers 22d1 and 22d2 and needle feature 12c in contact with base 22a of needle clip 22, so that needle clip 22 moves in unison with the proximal movement of needle 12. At this point, as barriers 22d1 and 22d2 are no longer biased by needle 12, hooks 22e3 and 22f3 would return to their natural position so as to disengage from flange 4d of catheter hub 4. For the embodiment shown in FIG. 8b, further movement of the needle in the proximal direction removes the needle clip from the catheter hub. FIG. 9 shows needle clip 22 housed inside clip guard 14 released from catheter hub 4 such that the catheter assembly is deemed to be in the safe state.

The catheter assembly with the needle clip embodiment shown in FIGS. 5-6 is further shown with reference to FIGS. 10a-10b, 11a-11b and 12a-12b. FIGS. 10a-10b show needle clip 22 housed inside clip guard 14 and catheter hub 4 being held by needle clip 22 in a ready or ready to use state. Clip guard 14 is removed from FIGS. 11a-11b and 12a-12b to illustrate with greater particularity the interaction between needle clip 22 and catheter hub 4. In each of FIGS. 11a-11b and 12a-12b, as barriers 22d1 and 22d2 are biased by needle 22, catheter hub 4 is coupled to needle clip 22. Since needle clip 22 and the proximal end portion of catheter hub 4 are covered by clip guard 14 as per shown in FIGS. 10a-10b, needle clip 22 is hidden from view from the time the catheter assembly is shipped in the ready state, during its use and to when the catheter hub is released from the needle clip to the disposal of the catheter assembly. After disposal, since needle clip 22 is housed within clip guard 14 and the distal portion of needle 12 including distal tip 12a is entrapped in needle clip 22, the distal tip of the needle is doubly prevented from being exposed to the environment.

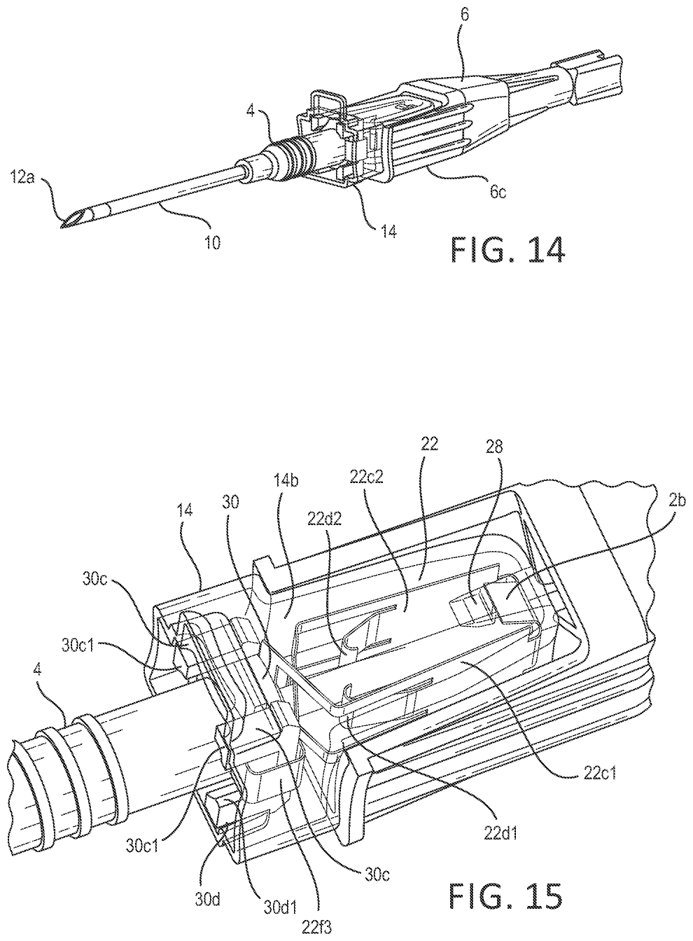

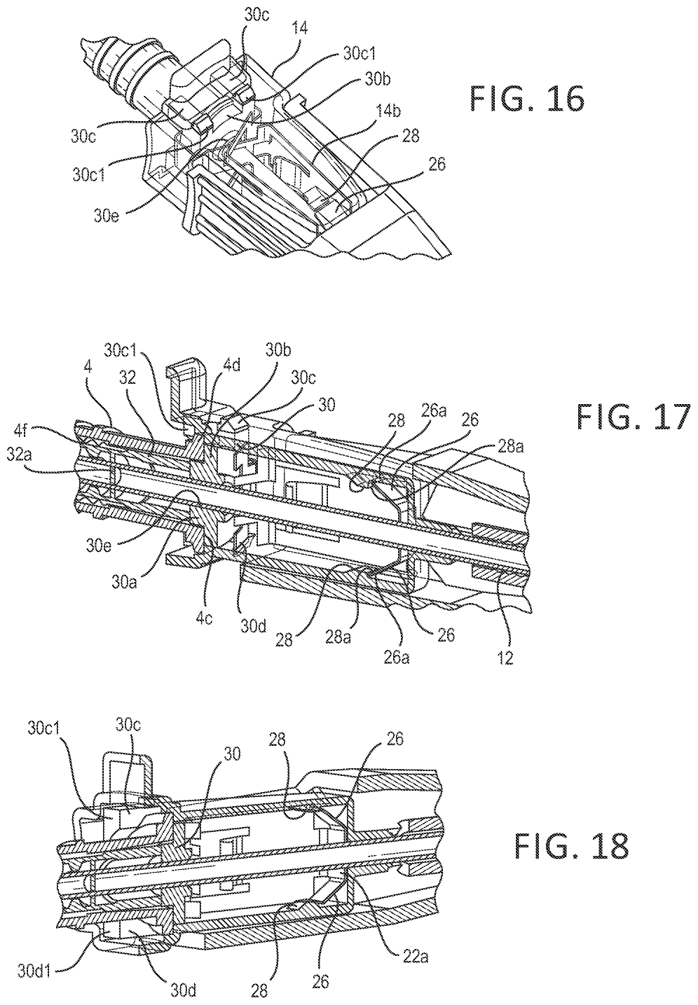

With reference to FIGS. 14-18, an alternative embodiment of the catheter assembly of the present invention is shown. In the alternate embodiment, components that are the same or function in the same manner as the earlier discussed embodiment are labeled with the same reference numbers. It should be appreciated that the exemplar catheter assembly shown in FIG. 14 is not the sideport catheter assembly as per shown in FIGS. 1-3. It should further be appreciated that the clip guard embodiment of FIG. 14 may be adapted to be used with different safety catheter assemblies, including the sideport catheter assembly shown in FIGS. 1-3 as well as a CSC assembly.

Similar to the earlier discussed catheter assembly, the FIG. 14 catheter assembly includes a catheter hub 4, a needle 12 having a distal tip 12a extending beyond the distal end of catheter 10. Catheter hub 4 is captured by a needle clip 22 in the same manner as discussed above, as for example per shown in FIG. 15. Needle clip 22 is housed in clip guard 14, which is shown semi-transparently. Clip guard 14 in turn is received in distal portion 6d of needle housing 6. Needle clip 22 has the same components as the needle clip described in FIGS. 5-6. To maintain needle clip 22 within chamber 14b of clip guard 14, a pair of opposing projections or tangs 26 extend distally at an angle from base 22a of needle clip 22. For the embodiment shown in FIG. 15, undercuts 28 or other structure adapted to coact with tangs 26 are formed at opposing inner walls of clip guard 14. When needle clip 22 is inserted into chamber 14b, tangs 26, when coming into contact with the tapered distal surfaces of undercuts 28, would bend inwardly to pass the undercuts. Once past the undercuts, tangs 26 revert back to their unbiased natural position so that their respective edges 26a would abut against the proximal back surfaces of the respective undercuts 28 to prevent needle clip 22 from moving in the distal direction relative to clip guard 14. As a result, needle clip 22 is non-removably housed in chamber 14b of clip guard 14. Note that the opposing tangs 26 extend from opposing sides of base 22a while the arms 22c1 and 22c2 of needle clip 22 extend from the other opposing sides of base 22a.

Further with respect to the embodiment shown in FIGS. 14-18, a wiper member 30 that is made from an elastomeric material is fitted to the opened proximal end 4c of catheter hub 4. Wiper member 30 may henceforth be referred to as a nose wiper, an elastomeric member or simply a wiper. Wiper 30, as best shown in the cross-sectional view of FIG. 17, has a distal portion or section 30a and a proximal portion or section 30b. Distal section 30a has a smaller dimension or cross section than the dimension of proximal section 30b. The distal surface of proximal section 30b abuts against most of the proximal end surface of the flange 4d at the proximal end of catheter hub 4. The cross dimension of distal section 30a is configured to enable distal section 30a to be press fitted to internal cavity 4f of catheter hub 4, so that distal section 30a may be frictionally held to the catheter hub.

As further shown in FIG. 17, wiper 30 may also act as a retainer to prevent a seal member 32 positioned in internal cavity 4f of catheter hub 4 from moving proximally relative to catheter hub 4. As shown, seal member 32 in FIG. 17 has a membrane 32a that reseals to prevent back flow of blood when needle 12 is removed from catheter hub 4. In place of a seal member, a resealable septum may be positioned inside internal cavity 4f of catheter hub 4 and be held thereat by wiper 30. If the catheter assembly of FIGS. 14-18 were a closed system catheter (CSC) assembly with an external port from the catheter hub but with no cylindrical sleeve covering the opening to the external port, the septum may be positioned in internal cavity 4f proximal the opening of the external port to prevent blood back flow to the portion of the internal cavity proximal the septum when needle 12 is withdrawn from catheter hub 4. While wiper 30 acts against the septum or seal member to prevent their movement in the proximal direction, an internal barrier for example an circumferential protrusion or shoulder at the inner wall of the catheter hub distal of and in abutment with the septum or seal member prevents the septum or seal member from moving in the distal direction. The septum, seal member and wiper are all made from elastomeric materials including for example silicone and polyisoprene, and each are formed as a unitary or monolithic component through various molding process including for example injection molding processes that are conventionally known.

Further with respect to FIGS. 15-18, wiper 30 is shown to have two upper extensions or arms 30c that extend from the top of proximal portion 30b and two lower extensions or arms 30d that extend from the bottom of proximal portion 30b. A hook or finger 30c1 is formed at the distal end of each of the upper arms 30c, and a hook or finger 30d1 is formed at the distal end of each of the lower arms 30d. The hooks 30c1 and 30d1 are configured to be in engagement with respective portions of the distal surfaces of flange 4d such that wiper 30 is firmly held to catheter hub 4. An opening or orifice 30e is provided in wiper 30 to enable needle 12 to pass therethrough. The dimension of orifice 30e is designed to be slightly smaller than the diameter of the needle so that the needle is in circumferential contact with the wall of orifice 30e so that fluid such as blood that may have adhered to the needle is wiped off as needle 12 is withdrawn from catheter hub 4. Thus, for the embodiment of FIGS. 14-18, needle 12 passes trough the bore of shaft 14e of clip guard 14 (FIG. 4b), the opening or aperture 22b at base 22a of needle clip 22, and orifice 30e of wiper 30 in the ready state. As discussed earlier, when needle 12 is withdrawn in the proximal direction relative to catheter hub 4 and distal tip 12a of needle 12 is captured by the barriers 22d1 and 22d2 inside needle clip 22, the hooks or hook/end combination of needle clip 22 are released from the flange 4d of catheter hub 4 so that clip guard 14 with needle clip 22 non-removably housed in its chamber 14b is separable from catheter hub 4. Once separated from the catheter hub 4, clip guard 14 is connected to needle housing 6 by the shaft of needle 12, and the entrapped needle of the catheter assembly is deemed to be in the safe state or safe position.

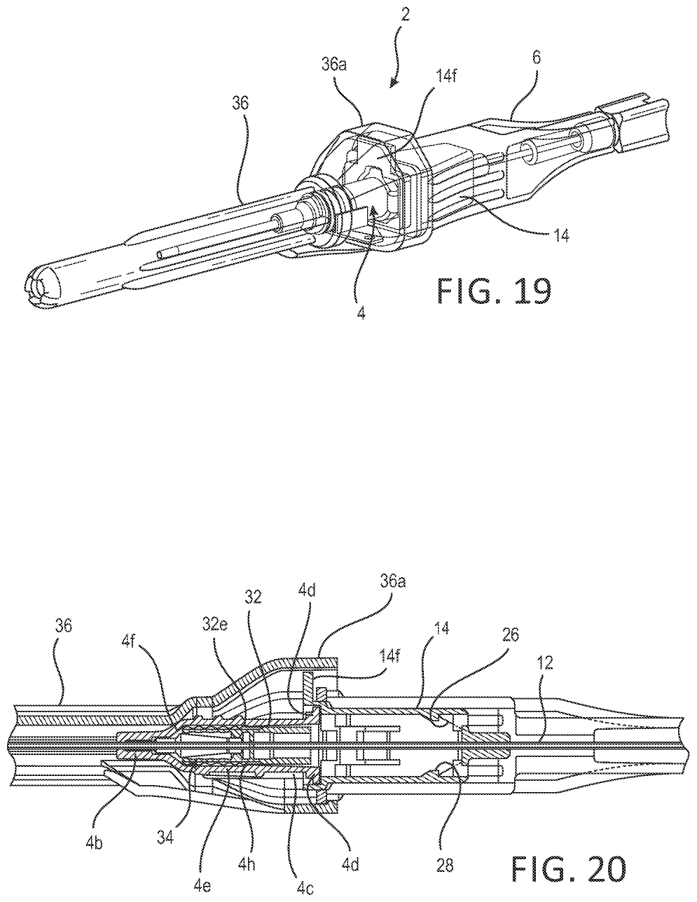

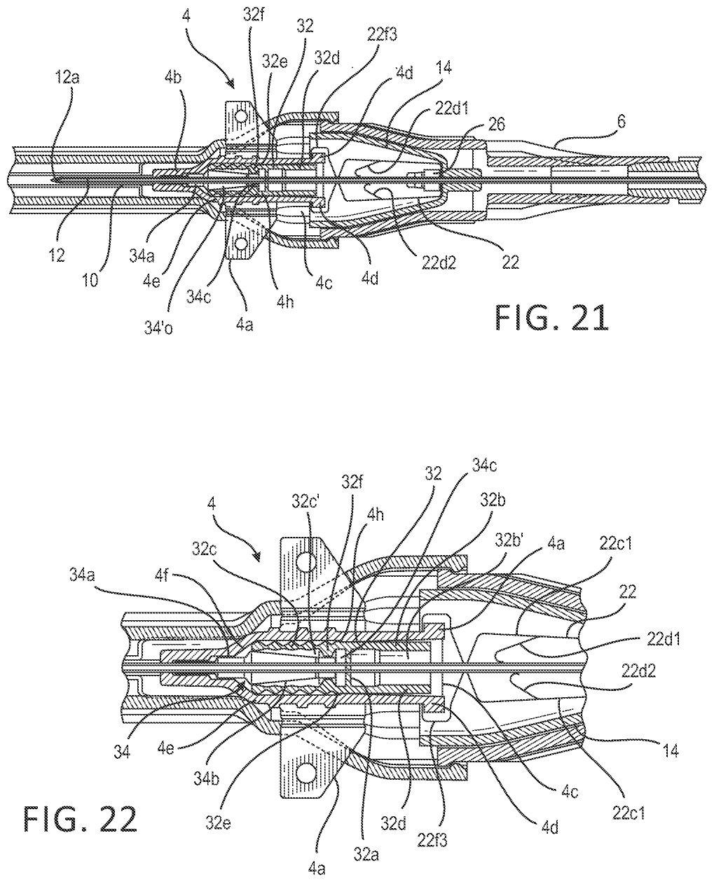

Another embodiment of the present invention safety catheter assembly is shown with reference to FIGS. 19-22. As is with the earlier embodiments, components that are the same or perform the same functions are labeled with the same reference numbers as above. The catheter assembly of FIGS. 19-22 does not have a wiper, and is not a sideport catheter assembly or a CSC assembly.

As shown in FIGS. 19-22, the catheter assembly has a catheter hub 4 that has side wings 4a and at least opposing flanges 4d at it proximal end 4c. A catheter 10 extends from distal end 4b of catheter hub 4, and a needle 12 having a sharp distal tip 12a extends beyond the distal end of catheter 10. Also as discussed above, wall 4e of catheter hub 4 defines an internal cavity 4f that has an opened proximal end 4c. For the embodiment of FIGS. 19-22, a multiuse seal member 32 (also shown in FIGS. 17-18) is provided within internal cavity 4f. No wiper is provided at opened proximal end 4c of catheter hub 4.

Seal member 32 is a substantially cylindrical multiuse valve shown to include a proximal cylindrical portion 32b and a distal cylindrical portion 32c. The portion of wall 32d that defines proximal portion 32b is substantially uniform along its length, whereas the portion of wall 32d that defines distal portion 32c has a corrugated shape and may be in the shape of an accordion along most if not all of its length. Membrane 32a is integral of seal member 32 and partitions internal cavity 4f of catheter hub 4 into respective bores of proximal portion 32b and distal portion 32c. When fitted into the interior cavity 4f of catheter hub 4, given that the seal member 32 is made of an elastomeric material such as silicone or polyisoprene, accordion shaped distal portion 32c is compressible when proximal portion 32b is under pressure or its proximal end is impacted by a distal driving force. Distal portion 32c and would return to its natural position when the pressure or impact force is removed from proximal end 4c. Although shiftable axially, seal member 32 is non-removably secured to the interior cavity 4f of catheter hub 4, as an internal circumferential flange or shoulder 32e may be provided at approximately the juncture between proximal portion 32b and distal portion 32c to catchingly engage the step 4h at interior cavity 4f of catheter hub 4. Step 4h prevents seal member 32 from proximal movement out of catheter hub 4 once seal member 32 is fittingly inserted and seated into interior cavity 4f by way of opened proximal end 4c of catheter hub 4.

As shown in FIGS. 20-22, provided inside interior cavity 4f and distal of membrane 32a is an actuator 34. The exemplar actuator shown in FIGS. 20-22 is similar to and functions substantially the same as the actuator described in the aforenoted incorporated by reference '104 US patent. Actuator 34 is shown to have a distal portion 34a that frictionally fits to the bore at the distal portion of catheter hub 4 and an enlarged proximal frusto-conical portion 34b that ends with an opened proximal end 34c distal of membrane 32a. The diameter of opening 34c is smaller than the diameter across frusto-conical portion 34b. Both distal portion 34a and proximal portion 34b are hollow so that a through passage is established between catheter 10 and opened proximal end 34c. The proximal end of the frusto-conical portion 34b is movably and sealingly held by an inwardly protruding circumferential protrusion 32f of seal member 32. By having frusto-conical section 34b, membrane 32a of seal member 32 may be more readily pierced or opened when seal member 32 is driven to shift axially in the distal direction by an impact force such as that applied when the luer connector of a syringe or other similar connector is connected to proximal end 34c via flange 4d, as described in the '104 US patent. When the connector and therefore the impact force is removed, seal member would return to its natural position due to the compressed accordion distal portion 32c decompressing. Membrane 32a reseals when it no longer is biased by opened proximal end 34c of actuator 34.

The coupling of catheter hub 4 with needle clip 22 is done in the same manner as discussed above, in that opposing flanges 4d (or portions of one flange) are grasped by hooks 22f3 at the distal ends of arms 22c1 and 22c2 of needle clip 22. Other than hooks are provided at both distal ends of the arms (instead of a combination of hook/end as shown in FIGS. 13a-13b), the coupling and decoupling of needle clip 22 and catheter hub 4 are the same as discussed above. Briefly, once distal tip 12a of needle 12 is captured in needle clip 22 proximal to barriers 22d1 and 22d2, hooks 22f3 of needle clip 22 are released from flange 4d of catheter hub 4 so that further proximal movement of needle 12 relative to catheter hub 4 would cause needle clip and clip guard 14 to separate from catheter hub 4. Needle clip 22 and clip guard 14 move in unison proximally from catheter hub 4 since needle clip 22 is non-removably housed in clip guard 14 due to the interaction of tangs 26 of needle clip 22 and undercuts 28 of needle clip 22 as discussed above.

The embodiment of FIGS. 19-22 has a sheath 36 that covers the catheter hub and the distal portion of needle housing 6 when clip guard 14 is received in the ready state. As shown, sheath 36 has an enlarged semi-funnel shaped proximal portion having an internal dimension adapted to be frictionally held by the distal portion of clip guard 14 by means of its upright 14f and the respective planar side surfaces and the bottom surface of clip guard 14. It should be noted that the proximal end configuration of the clip guard 14 shown in FIGS. 19-22 is slightly different from the clip guard shown in FIGS. 10a-10b, in particular with respect to the positioning of upright 14f. Sheath 36 prevents damage to the catheter hub, the catheter and the needle during transit and is removed when the catheter assembly is to be used.

It is the intension of the inventors that all matter described throughout this specification and shown in the accompanying drawings be interpreted as illustrative only and not in a limiting sense. Accordingly, it is intended that the invention be limited only by the spirit and scope of the hereto appended claims.

1. A catheter assembly comprising:
a catheter hub having a body defined by a wall having a distal end and an opened proximal end having a flange, and an internal cavity defined by an inner surface of the wall between the distal end and the opened proximal end;
a catheter having a distal end extending from the distal end of the catheter hub along a longitudinal axis;
a wiper member including a distal section having a cross dimension configured to fit into the opened proximal end of the catheter hub into the internal cavity and a proximal section having a cross dimension larger than the cross dimension of the distal section;
a guard in alignment with a needle hub along a longitudinal axis, the guard configured as a chamber having an open distal end and a proximal base having an aperture;
a needle defined by a cannula shaft having a proximal end attached to the needle hub, the shaft of the needle extending through the aperture at the base of the guard such that the needle is slidable relative to the guard, the needle including a sharp distal tip extending away from the open distal end of the guard along the longitudinal axis such that the needle slidably extends along the catheter with the distal tip extending beyond the distal end of the catheter when the catheter assembly is in a ready to use position;
a needle tip protector slidably positioned along the needle;
wherein the chamber is configured to house the needle tip protector.

2. The catheter assembly of claim 1, wherein the distal section of the wiper member is pressure fitted to the opened proximal end of the catheter hub, and wherein the proximal section of the wiper member comprises at least opposing hook members that extend from the proximal section distally over and latches onto a distal surface of the flange to fixedly hold the wiper member to the catheter hub.

3. The catheter assembly of claim 2, wherein the guard comprises a distal opening defined by an inner surface configured to contact respective outer surfaces of the hook members such that the guard is in frictional engagement with the wiper member to prevent free play between the guard and the catheter hub when the catheter assembly is in the ready to use position.

4. The catheter assembly of claim 3, wherein the guard comprises an upright adapted to be pushed by a user for inserting the distal tip of the needle and a distal portion of the catheter into a vein or artery of a subject.

5. The catheter assembly of claim 1, wherein the needle tip protector comprises a base and two arms extending from the base, the arms having respective distal portions that crisscross each other, respective naturally inward biasing barriers extending from the arms proximal of the distal portions, openings at the distal portions enable the needle to pass through, a finger provided at the distal end of each of the distal portions adapted to grasp the flange at the proximal end of the catheter hub.

6. The catheter assembly of claim 5, wherein the barriers of the needle tip protector are biased by the needle passing through the openings at the distal portions to cause the finger at the distal end of each of the arms to grasp the flange to removably couple the guard and the catheter hub to each other.

7. The catheter assembly of claim 1, further comprising a sideport extending from the catheter hub to establish a through passage between the internal cavity of the catheter hub and an open end of the sideport via a sideport opening at the wall of the catheter hub.

8. The catheter assembly of claim 7, further comprising a resealable valve covering the sideport opening mounted to the inner wall of the catheter hub, the sideport opening adapted to be opened when subjected to an input fluid at a given pressure or when push opened by a tubing inserted through the open end of the sideport hub.

9. The catheter assembly of claim 1, wherein the needle shaft defines a lumen and has a side orifice proximate to its distal tip to enable fluid to flow into the lumen and egress at the orifice into a space defined between the outer wall of the needle and the inner wall of the catheter when the distal end of the needle is inserted into a subject.

10. The catheter assembly of claim 1, wherein the chamber at the guard comprises engagement mechanism for non-removably holding the tip protector within the guard from the ready to use position to a safe position where the distal tip of the needle is captured proximal the barriers in the needle tip protector such that the unbiased barriers return to their natural position to cause the fingers to be removed from the flange to enable the guard to be separated from the catheter hub.

11. The catheter assembly of claim 1, further comprising a resealable septum positioned in the internal cavity of the catheter hub distal the wiper member, the septum pierced by the needle when the catheter assembly is in the ready to use position, the septum reseals to close off the internal cavity distal of the septum from the proximal end of the catheter hub when the needle is withdrawn from the septum.

12. A catheter assembly comprising:
a catheter hub having a body defined by a wall having a distal end and an opened proximal end having a flange, and an internal cavity defined by an inner surface of the wall between the distal end and the opened proximal end;
a catheter having a distal end extending from the distal end of the catheter hub along a longitudinal axis;
a wiper member including a distal section having a cross dimension configured to fit into the opened proximal end of the catheter hub into the internal cavity and a proximal section having a cross dimension larger than the cross dimension of the distal section and an orifice therethrough;
a needle having a sharp distal tip extending through the orifice of the wiper member and slidably extending along the catheter with the distal tip extending beyond the distal end of the catheter when the catheter assembly is in a ready to use position.

13. The catheter assembly of claim 12, further comprising a needle tip protector slidable along the needle, the needle tip protector having a base with an opening through which the needle passes, the needle tip protector including barriers biased by the needle and fingers responsive to the barriers being biased by the needle to grasp the catheter hub when the needle passes between the barriers.

14. The catheter assembly of claim 13, further comprising a guard having an open distal end and a proximal base having an aperture through which the needle passes, the needle tip protector received into a chamber of the guard that opens to the open distal end, the needle passing through the aperture of the guard, the opening at the base of the needle tip protector and the orifice of the wiper member when the catheter assembly is in the ready to use position.

15. The catheter assembly of claim 12, wherein the catheter hub includes a sideport hub having an opened end and an opening into the internal cavity of the catheter hub to establish a through fluid passage between the internal cavity of the catheter hub and a tubing adapted to be connected to the opened end of the sideport hub.

16. The catheter assembly of claim 15, further comprising a resealable valve mounted to the inner wall of the catheter hub to cover the sideport opening, the sideport opening adapted to be opened when subjected to an input fluid at a given pressure or when push opened by a tube inserted through the opened end of the sideport hub.

17. The catheter assembly of claim 12, further comprising a needle housing having a distal opening adapted to receive the guard when the needle assembly is in the ready to use position, the needle housing having a needle hub proximal the distal opening to which a proximal end of the needle is fixedly attached.

18. The catheter assembly of claim 14, wherein the chamber at the guard comprises engagement mechanism for non-removably holding the needle tip protector within the guard from the ready to use position to a safe position where the distal tip of the needle is captured proximal the barriers in the needle tip protector, the guard separable from the catheter hub and slidably along the needle away from the needle housing in the safe position.

19. The catheter assembly of claim 18, wherein the engagement mechanism comprises at least one undercut inside the chamber of the clip guard that receives a counterpart tang extending from the base of the needle tip protector to prevent the needle tip protector from exiting the guard once the guard is fully inserted into the chamber.

20. The catheter assembly of claim 12, further comprising a resealable septum positioned in the internal cavity of the catheter hub distal the wiper member, the septum pierced by the needle when the catheter assembly is in the ready to use position, the septum reseals to close off the internal cavity distal of the septum from the proximal end of the catheter hub when the needle is withdrawn from the septum.
